(19) United States
(12) Patent Application Publication
(10) Pub. No.: US 2007/0061017 A1
(43) Pub. Date: Mar. 15, 2007

(54) Liner for use with prosthetic devices

(76) Inventor: Michael T. Wilson, Missouri City, TX (US)

Correspondence Address: CONLEY ROSE, P.C., P. O. BOX 3267, HOUSTON, TX, 77253-3267, US

(21) Appl. No.: 11/223,507

(22) Filed: Sep. 9, 2005

(51) Int. Cl. A61F 2/78

(52) U.S. Cl. 623/36

(57) ABSTRACT

A liner for use with a prosthesis has a height and an area and comprises an optional inner layer, a cushion layer affixed to the inner layer, an outer layer affixed to the cushion layer, and an optional bead affixed to at least one of the layers and defining the upper edge of the liner, wherein the cushion layer has an area that is less than less than 90% of the area of the liner. The cushion layer may extend from the upper edge of the liner or from the bottom of the liner to a point between the top and bottom edges, or may be present only desired portions of the liner area.

### CROSS-REFERENCE TO RELATED APPLICATIONS

Not Applicable.

### STATEMENT REGARDING FEDERALLY SPONSORED RESEARCH OR DEVELOPMENT

Not Applicable.

### FIELD OF THE INVENTION

The present invention relates generally to liners for use with prosthetic devices and more particularly to a method for manufacturing liners having an improved proximal edge.

### BACKGROUND OF THE INVENTION

Prosthesis liners formed from silicone elastomeric materials have been described in prior patents, such as, for example, U.S. Pat. Nos. 4,923,474, 5,507,834, 5,376,129, 5,658,354, 5,888,216, 6,136,039, and 6,508,842. Elastomeric liners are commonly used to cushion a post-operative stump or residual limb with respect to a prosthesis. The prosthesis will typically include a rigid socket that is shaped to receive the stump or residual limb. The socket is installed over the residual limb and in some instances may be coupled to the liner by a locking element.

Partial leg amputations in which the leg is amputated at the thigh level are typically referred to as “transfemoral,” “above knee,” or “AK” amputations and will hereinafter be referred to as TF amputations. Likewise, partial leg amputations in which the leg is amputated below the knee are typically referred to as “transtibial,” “below knee,” or “BK” amputations and will hereinafter be referred to as BK amputations. In BK amputations, the patient's knee joint remains viable and the prosthesis is constructed so that the mechanical load of the patient's body is transferred through the knee joint to the prosthesis. For this reason, it is important to provide significant cushioning at the bottom of a BK prosthesis liner. By contrast,

In both TF and BK amputations, the prosthesis itself is held in place through mechanical engagement between the socket and the liner and the liner is in turn held in place by air pressure or “suction.” Specifically, with the partial limb in place within the liner, the upper edge of the liner is sealed against the limb. This prevents air from entering the space between the limb and the liner and prevents the liner from falling off. For this reason, the term “suction socket” is occasionally used. Because sealing the liner to the limb often requires applying pressure around the upper edge of the liner, it can cause extreme discomfort if the upper edge of the liner is not constructed with this consideration in mind.

In both TF and BK prostheses, it is desirable that such liners conform to the outer surface of the residual limb and provide a comfortable cushion between the residual limb and the rigid socket of the prosthesis that is to be fitted over the residual limb. Special silicone rubber or elastomeric materials have been formulated as suitable substances for prosthesis liners. Such materials are typically selected on the basis of one or more of the following properties: hardness (or softness), elongation, tensile strength, sterilizability, porosity, and ease of cleaning. In particular, composite materials including silicone have been used successfully for prosthesis liners.

Despite advances in the materials, certain aspects of prosthesis liners remain sub-optimal. One such aspect is the weight of the liner. Because the elastomeric materials that are used to provide cushioning are relatively dense, they add significantly to the overall weight of the prosthetic system. It is therefore desirable to avoid the use of cushioning materials in areas where it is not needed. Hence, it remains highly desirable to optimize placement of the cushion material within the prosthesis liner.

### SUMMARY OF THE INVENTION

The present invention provides a prosthesis liner, in which placement of the cushion material within the liner is optimized so as to provide optimum comfort to the wearer. In a liner for a TF prosthesis, the cushion material extends from the upper edge of the liner to a point well above the bottom of the liner. In a liner for a BK prosthesis, the cushion material extends across the bottom of the liner and partially up the sides of the liner but stops well below the upper edge of the liner.

Thus, the present invention comprises a combination of features and advantages that enable it to provide improved comfort and durability over previously known devices. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is an illustration showing an amputee with liner and prosthetic limb doffed;

FIG. 2 is a side view of a liner constructed in accordance with a first embodiment of the invention, showing the liner in partial cross-section; and

FIG. 3 is a side view of a liner constructed in accordance with a second embodiment of the invention, showing the liner in partial cross-section.

### DETAILED DESCRIPTION OF THE PREFERRED EMBODIMENT

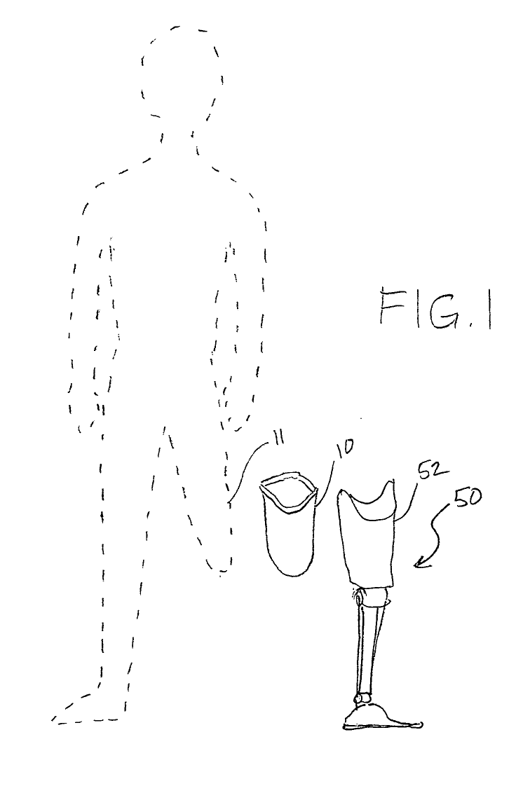

Referring initially to FIG. 1, an amputee who has had a limb partially removed will typically have a stump or “residual limb” 11, to which a prosthesis for the missing limb part can be attached. While FIG. 1 shows a transfemoral amputee and the discussion below may be presented in terms of a prosthetic leg, it will be understood that other embodiments of the present invention can be used advantageously in any partial limb amputation, including but not limited to transfemoral and transtibial amputations. In FIG. 1, the liner 10 and the prosthesis 50 are shown separated from the wearer. Prosthesis 50 includes a socket 52, which is typically rigid and custom-shaped to conform generally to the outer surface of the partial limb 11.

Regardless of whether a prosthesis is adapted to be used on a TF amputee, a BK amputee, or in place of a partially amputated arm instead of a leg, a liner used in conjunction with the prosthesis will be shaped to conform to the partial limb. As such, the liner will be essentially cup-shaped, with an “upper edge,” an “inside,” and an “outside.” As used herein, those terms are intended to have their conventional meanings and are intended to apply to the structure of the line without regard to its physical orientation during use. Likewise, because it is configured to fit over a three-dimensional shape (the partial limb), the liner has an “area,” which as used herein is defined as the surface area of the three-dimensional shape that is covered by the liner.

As stated above, liner 10 fits over the partial limb and forms a cushion between the partial limb 11 and socket 52. Thus, liner 10 can have a great effect on the level of comfort experienced by the wearer. Despite the need for its presence, however, the cushion layer adds bulk and weight to the prosthesis and in some respects can create as well as alleviate discomfort.

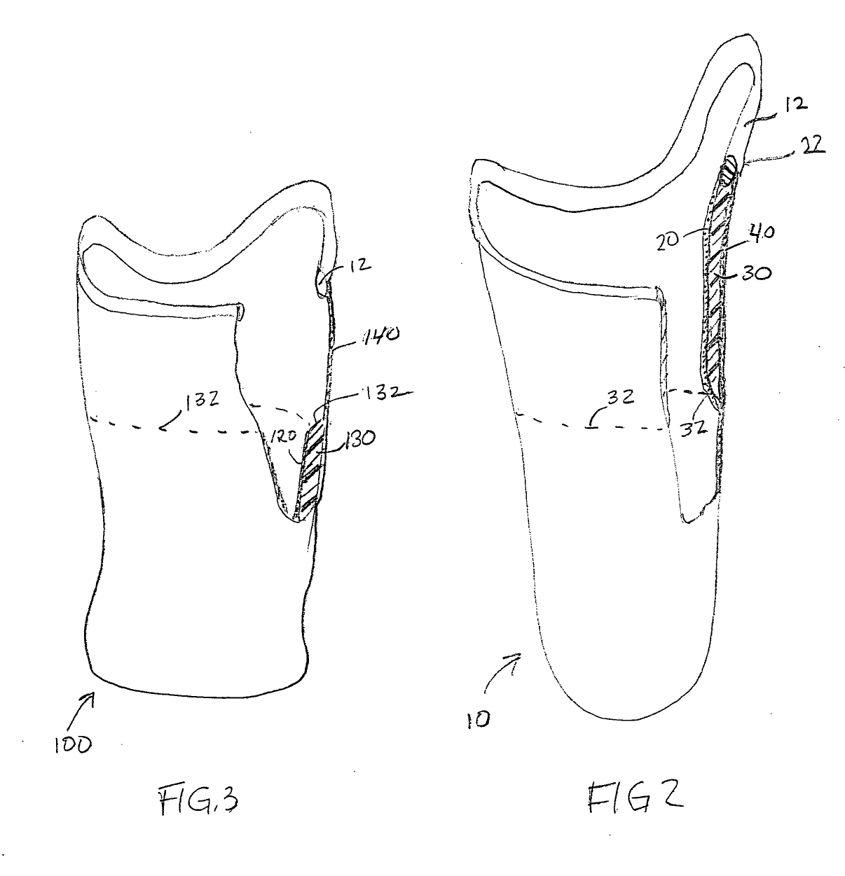

Thus, referring now to FIG. 2, a preferred embodiment of a liner 10 for a TF prosthesis includes a inner layer 20, a partial cushion layer 30, and an outer layer 40. Inner layer 20 and outer layer 40 each preferably comprise at least one layer of elastomer-impregnated fiber, such as knitted nylon mesh or the like impregnated with polyurethane, silicone, or the like, such as is known in the art. Cushion layer 30 is preferably elastomeric and preferably comprises a relatively thick layer of silicone gel. Alternatively, cushion layer 30 may comprise urethane rubber, expanded closed cell EVA, closed cell silicone sponge, or any other suitable material.

Prostheses for TF amputees are constructed so that the weight of the wearer is supported by the upper edge of the socket, upon which the wearer places his/her tailbone. The mechanical load is transmitted through the socket to the prosthetic limb. Thus, the amputee's partial limb does not bear a significant portion of the mechanical load. While including cushion layer 30 at the upper edge of the liner increases patient comfort, it has been found that the presence of cushion layer 30 in the lower half of the liner does little to increase comfort and may in fact increase discomfort, as it adds to the weight of the liner and impedes heat loss.

In the embodiment shown in FIG. 2, the cushion layer 30 extends from the upper end 22 of the liner approximately halfway down the length of the liner. Thus, depending on the size of the patient, the cushion layer extends downward at least 2 inches from the upper edge of the liner and preferably terminates between about 5 and about 10 inches from the upper edge. Below the lower edge 32 of cushion layer 30, inner layer 20 may terminate. If inner layer 20 extends beyond the extent of cushion layer 30, inner layer 20 and outer layer 40 will be in direct contact in that portion of the liner, as there is no cushion layer between them.

In some embodiments, a molded bead 12 is included around the upper edge of liner 10. Bead 12 may be positioned at least partially between inner layer 20 and outer layer 40. In other embodiments (not shown), bead 12 may be positioned inside inner layer 20 or outside outer layer 40. In still other embodiments, bead 12 may be disposed adjacent to the edges of inner and outer layers 20, 40. Bead 12 preferably comprises a continuous or joined loop of elastomeric material, such as silicone or urethane. The thickness of bead 12 may be varied along the length of the loop and can be designed to provide optimized comfort to the wearer. For example, it has been that wearer comfort can be enhanced when bead 12 is thicker at the portion of liner 10 that lies adjacent to the inner thigh of the wearer and thinner at the portion of liner 10 that lies adjacent to the outer thigh of the wearer. In some embodiments, bead 12 will range from 0.18 to 0.5 inch thick.

Turning now to FIG. 3, an embodiment of a liner 100 for a BK prosthesis includes a inner layer 120, a partial cushion layer 130, and an outer layer 140. As in the liner above, inner layer 120 and outer layer 140 each preferably comprise at least one layer of elastomer-impregnated fiber, such as knitted nylon mesh or the like impregnated with polyurethane, silicone, or the like, while cushion layer 130 is preferably comprises a relatively thick layer of a resilient elastomer.

As shown in FIG. 3, the cushion layer 130 extends from the bottom of the liner about halfway up the height of the liner. Thus, depending on the size of the patient, the cushion layer extends upward to a point that is at least 2 inches from the upper edge of the liner and preferably between about 5 and about 10 inches from the upper edge. Above the upper edge 132 of cushion layer 130, inner layer 120 and outer layer 140 are in direct contact, as there is no cushion layer between them.

In a BK prosthesis, the weight of the wearer is supported through the patient's knee, which bears on the upper end of the prosthesis. Thus, in a BK prosthesis, cushioning at the bottom of the socket is more important than it is in a TF prosthesis. Hence, in BK embodiments, the cushion layer is provided at the bottom of the liner and eliminated from the upper portion of the liner.

It has been found that patient comfort can be increased in a BK prosthesis if the upper edge of the liner is flared slightly so as to gradually decrease pressure on the partial limb. It may also be advantageous to provide a bead 12 as described above.

It has been discovered that by applying the concepts disclosed herein, the weight of the liner can be reduced by more than ten percent, in some instances by more than 25 percent and in some instances by more than 40 percent.

It will be understood that it is not necessary for both the inner and outer layers to extend the full height of the liner. In some embodiments, either the inner layer or outer layer may extend only as far as the extension of the cushion layer. Thus, for example, a liner may have an outer layer that extends to the full height of the liner and a cushion layer and inner layer that each terminate at approximately half the height of the liner. As described above, the partial cushion (and partial liner if desired) can extend from either the top or the bottom of the liner.

In other embodiments, the partial cushion layer may be placed in the middle portion of the liner, or may be divided so as to provide cushioning in desired regions of the liner. In such embodiments, it has been found advantageous to configure the cushion layer such that covers less than 90% of the area of the liner, preferably less than 80%, and still more preferably less than 70%.

While any suitable method can be used to assemble a liner 10 in accordance with the present invention and having the desired traits of comfort and durability, a preferred method is as follows. First, a plaster cast of the wearer's stump or residual limb is taken and used to make plaster positive model. Next inner layer 20 is applied to the outside of the positive model. Inner layer 20 preferably comprises a laminated composite layer and may be formed by any suitable means. In one embodiment, one, two or more layers of stockinet comprising two-way stretch nylon, glass fiber or other suitable material are used for inner layer 20. This fiber layer is enclosed in an airtight film, which is then evacuated. The matrix portion of the composite inner layer, such as liquid silicone, is introduced to the stockinet layer and is forced by atmospheric pressure to flow into and impregnate the stockinet. Once the stockinet is fully impregnated, the airtight film is removed.

Bead 12 is applied to the desired configuration of the upper edge of the liner, preferably using silicone glue. A transfer layer such as tape or another transfer means is then applied over inner layer 20, the desired upper edge of the liner 10 is marked on the transfer means, and the transfer means is removed. The removed transfer means is cut and at least partially flattened on a substantially flat molding surface and the partially flattened layer is outlined on the molding surface to so as form a pattern for the cushion layer of the liner.

The transfer means is then discarded and a mold edge or containment wall is used to outline the pattern. This edge defines the desired edge of the cushion layer. In combination with the flattened tape layer, the edge forms a mold for construction of the cushion layer. If it is desired to fill less than all of the mold with cushioning material, as in the case of the present partial cushion layers, the portion of the mold that is to be unused may be blocked or filled by any suitable means.

Once the desired mold is configured, liquid silicon is poured into it, preferably to a thickness of about ⅛ inch. In preferred embodiments, a parting agent or film is laid over the silicone and the mold is flattened between parallel plates to ensure a uniform thickness. The resulting cured material forms partial cushion layer 30, 130. In alternative embodiments, the cushion layer can be formed by any suitable means. For example, a negative corresponding to the inner surface of the socket could be provided. When the positive coated with the inner layer is placed in the negative, the cushioning material can be poured or injected into a clearance between the two. Other techniques for forming the cushion layer will be known to those skilled in the art.

Once the partial silicone cushion layer 30, 130 has cured, it is removed from the mold. If a bead 12 is desired and has not already been applied to inner layer 20, bead 12 is affixed in the desired position on inner layer 12. Partial cushion layer 30, 130 is then shaped and wrapped around the outside of inner layer 20 and glued in place. During this process, the edges of cushion layer 30, 130 are brought up to bead 12 so that a continuous cushion layer is defined. If cushion layer 30, 130 is formed from a pattern that is slightly smaller than the desired surface, it can be slightly stretched as it is affixed to inner layer 20, ensuring a smooth and even fit.

Outer layer 40 is applied to the outer surface of the cushion layer. In preferred embodiments, outer layer 40 comprises another layer of fiber such as stockinet applied to the outside of the cushion layer, enclosed in a removable airtight layer, and impregnated with liquid silicone as described above.

When a liner 10 has been constructed in accordance with the present invention, bead 12 will define the finished dimension, shape, contour and texture of the proximal or upper edge of the liner. Because it is designed for this precise purpose, is provides superior comfort over previously known liner technologies.

It is preferred to use urethane or silicone for the construction of all layers of liner 10, in part because those materials can be doped with mineral oil, vitamin E, and/or other additives that improve wearer comfort, and also because those two materials do not lend themselves to use with other adhesives.

While preferred embodiments of this invention have been shown and described, modifications thereof can be made by one skilled in the art without departing from the scope or teaching of this invention. For example, the materials from which the components of the present liner are made may be varied, the shape and configuration of the finished liner may be varied, and the steps used in constructing the liner may be modified. Accordingly, the scope of protection is not limited to the embodiments described herein, but is only limited by the claims that follow, the scope of which shall include all equivalents of the subject matter of the claims. Likewise, the sequential recitation of steps in a claim, unless explicitly so stated, is not intended to require that the steps be performed in any particular order or that a particular step be completed before commencement of another step.

What is claimed is:

1. A liner for use with a prosthesis, said liner having a height and an area and comprising:
optionally, an inner layer;
a cushion layer affixed to said inner layer;
an outer layer affixed to said cushion layer; and
optionally, a bead affixed to at least one of said layers and defining the upper edge of the liner;
wherein said cushion layer has an area that is less than less than 90% of the area of the liner.

2. The liner of claim 1 wherein said cushion layer has an area that is less than 80% of the area of the liner.

3. The liner of claim 1 wherein said cushion layer has an area that is less than 70% of the area of the liner.

4. The liner of claim 1 wherein said cushion layer has a vertical extension that is less than 80% of the height of the liner.

5. The liner of claim 1 wherein said cushion layer has a vertical extension that is less than 60% of the height of the liner.

6. The liner of claim 1 wherein said cushion layer has a vertical extension that is less than 70% of the height of the liner.

7. The liner of claim 1 wherein said cushion layer extends from the upper edge of the liner to a point that is between about 5 and about 10 inches from said upper edge.

8. The liner of claim 1 wherein said cushion layer extends from the bottom of the liner to a point that is between about 5 and about 10 inches from said upper edge.

9. The liner of claim 1 wherein at least one of said inner layer and said outer layer has an area that is no greater than the area of said cushion layer.
